(19) United States
(12) Patent Application Publication
(10) Pub. No.: US 2024/0148176 A1
(43) Pub. Date: May 9, 2024

(54) Chenille Carpet

(71) Applicant: Tonglu Yongsheng Knitting Machinery Co. Ltd, Hangzhou City (CN)

(72) Inventor: Wenzheng Zheng, Hangzhou City (CN)

(21) Appl. No.: 18/407,418

(22) Filed: Jan. 8, 2024

(30) Foreign Application Priority Data
Oct. 31, 2023 (CN) CN202311443904.8

(51) Int. Cl. A47G 27/02; B32B 3/12; B32B 5/02; B32B 5/26; B32B 7/09; B32B 27/12; B32B 27/34

(52) U.S. Cl. CPC A47G 27/0243; B32B 3/12; B32B 5/024; B32B 5/028; B32B 5/262; B32B 7/09; B32B 27/12; B32B 27/34; B32B 2262/0276; B32B 2262/065; B32B 2262/16; B32B 2307/56; B32B 2307/724; B32B 2471/02

(57) ABSTRACT

The present disclosure discloses a chenille carpet, comprising a three-dimensional breathable base layer (1), wherein a three-dimensional weaved layer (2) made of chenille threads is provided on the three-dimensional breathable base layer (1), and the chenille threads in the three-dimensional weaved layer (2) have a diameter not less than 1 cm. The chenille carpet according to the present disclosure has high breathability, is not easy to trap dirt, and is easy to clean.

### CROSS-REFERENCE TO RELATED APPLICATION

This application claims priority to Chinese Patent Application No. 202311443934.8, filed on Oct. 31, 2023 before the China National Intellectual Property Administration, the disclosure of which is incorporated herein by reference in entirety.

### TECHNICAL FIELD

The present disclosure relates to the field of carpet or rug, and in particular to a chenille carpet.

### BACKGROUND

Chenille material is a high-performance synthetic fiber material, with good elasticity and adaptability. It is soft and comfortable, has a very good touch, and the chenille material has excellent wear resistance and can withstand long-term use and frequent wash, without obvious wear or damage, especially its chenille yarn has good softness and fineness, therefore now people are beginning to apply it to carpets, so that the carpets can give people a more comfortable experience.

Chenille carpets currently on the market are usually formed by tufting chenille yarn on a base fabric using a tufting loom. The base fabric has uniform and neatly arranged spike-like piles, but the chenille carpets are usually highly hygroscopic. When laid on the ground, they easily absorb ground moisture, causing the carpets to become mildewed, affecting the service life of the carpets. Moreover, the tufted piles have longer suede, it easily traps dirt, making it relatively difficult to clean, further causing damage to the carpets.

### SUMMARY

The object of the present disclosure is to provide a chenille carpet, which has high breathability, is not easy to trap dirt, and is easy to clean.

The technical solutions of the present disclosure include:

A chenille carpet, comprising a three-dimensional breathable base layer, wherein a three-dimensional weaved layer made of chenille threads is provided on the three-dimensional breathable base layer, and the chenille threads in the three dimensional weaved layer have a diameter not less than 1 cm.

According to some embodiments of the present disclosure, the three-dimensional weaved layer is weaved by plain knitting needles using chenille threads.

According to some embodiments of the present disclosure, the three-dimensional breathable base layer comprises a bottom layer, a three-dimensional support layer provided on the bottom layer, and a top layer provided on the three-dimensional support layer, a hemming is provided on an edge of the base layer, and the bottom layer and the top layer are mesh cloth.

According to some embodiments of the present disclosure, a mesh size of the bottom layer is less than a mesh size of the top layer, and the bottom layer is provided with evenly distributed glue dots at its bottom surface.

According to some embodiments of the present disclosure, the three-dimensional support layer is made of nylon coils with lower and upper ends connected to the bottom layer and the top layer respectively.

According to some embodiments of the present disclosure, the hemming comprises an inner layer made of linen material and an outer layer provided on the inner layer and made of polyester fabrics or polyester super soft fabric material.

According to some embodiments of the present disclosure, the three-dimensional breathable base layer and the three-dimensional weaved layer are detachably connected.

According to some embodiments of the present disclosure, the three-dimensional breathable base layer and the three-dimensional weaved layer are fixedly connected.

Compared with the prior art, the present disclosure has the following advantages:

I. In the present disclosure, it is provided with a three-dimensional weaved layer of chenille threads. The three-dimensional weaved layer of chenille threads is relatively soft and comfortable, and the diameter of the chenille threads is not less than 1 cm. When the chenille threads are interlaced/weaved, gaps will be formed, which not only improves the breathability, but also increases the height of the carpet surface layer, making the feet feel softer.

II. The three-dimensional weaved layer in the present disclosure is made of chenille threads by plain knitting needles. It can not only improve the breathability of the carpet surface layer, but also make it softer. The three-dimensional weaved layer is made in a way of plain knitting needle, and the diameter of the chenille threads is larger, therefore larger pores will be produced, and the debris falling on the carpet surface layer can be easily poured out, making it easy to clean.

III. There is a three-dimensional breathable base layer under the three-dimensional weaved layer of chenille threads in the present disclosure. The three-dimensional support layer in the three-dimensional breathable base layer is made of nylon coils, which not only makes the breathability better, but also makes the three-dimensional weaved layer of chenille threads separate from the ground farther away, so that the moisture and water vapor on the ground would not easily affect the three-dimensional weaved layer of chenille threads.

### BRIEF DESCRIPTION OF THE DRAWINGS

FIG. 1 is a schematic structural view of the present disclosure;

FIG. 2 is a schematic structural view of the present disclosure;

FIG. 3 is a schematic structural view of the three-dimensional weaved layer of chenille threads according to the present disclosure;

FIG. 4 is a schematic structural view of the top layer of the present disclosure;

FIG. 5 is a schematic structural view of the bottom layer of the present disclosure.

Reference number: 1—three-dimensional breathable base layer, 2—three-dimensional weaved layer of chenille threads, 1.1—bottom layer, 1.2—three-dimensional support layer, 1.3—top layer, 1.5—glue dot, 1.7—inner layer, 1.8—hemming/covered edge, 1.9—outer layer.

### DETAILED DESCRIPTION OF EMBODIMENTS

The technical solutions of the present disclosure will be further described in detail below with reference to embodiments and in conjunction with the accompanying drawings.

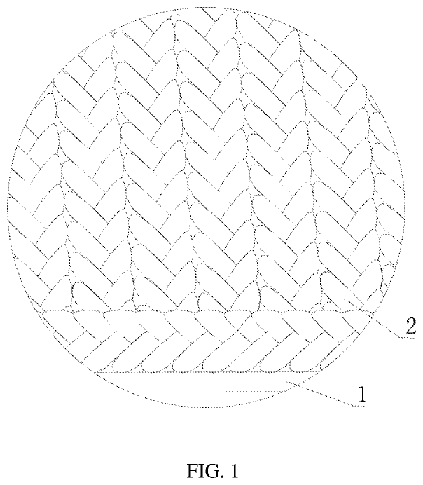

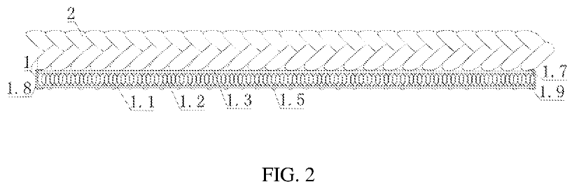

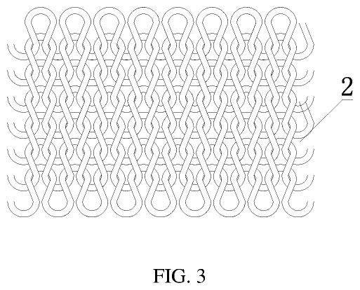

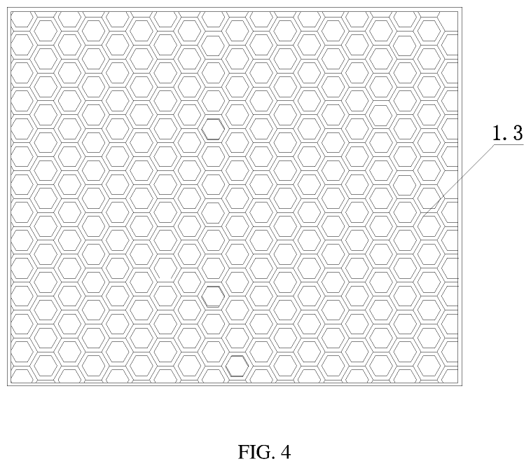

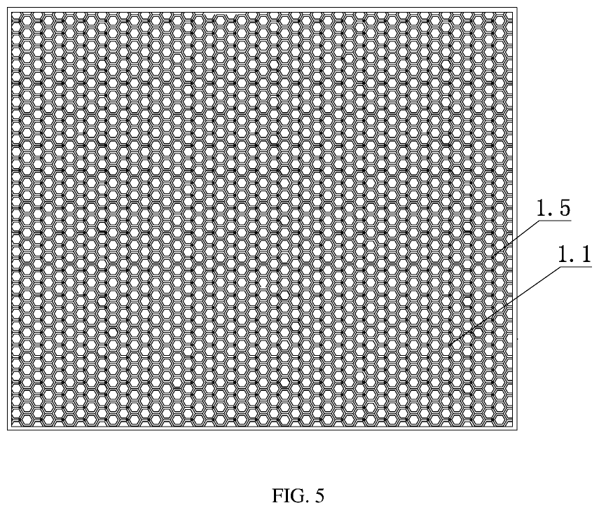

Embodiment 1: A chenille carpet, as shown in FIGS. 1-5, includes a three-dimensional breathable base layer 1. A three-dimensional weaved layer 2 of chenille threads is provided on the three-dimensional breathable base layer 1. The chenille threads in the three-dimensional weaved layer 2 has a diameter not less than 1 cm.

The three-dimensional weaved layer 2 of chenille threads is weaved and made of chenille threads, knitted with plain knitting needles/weft knitting needles.

The three-dimensional breathable base layer 1 includes a bottom layer 1.1, a three-dimensional support layer 1.2 provided on the upper side of the bottom layer 1.1, and a top layer 1.3 provided on the upper side of the three-dimensional support layer 1.2, hemmings/covered edges 1.8 are provided on the edges of the base layer 1, and the bottom layer 1.1 and the top layer 1.3 are mesh cloth.

The mesh size of the bottom layer 1.1 is smaller than the mesh size of the top layer 1.3, and evenly distributed glue dots 1.5 are provided on the lower side of the bottom layer 1.1.

The three-dimensional support layer 1.2 is made of nylon coils, with lower and upper ends are connected to the bottom layer 1.1 and the top layer 1.3 respectively.

The hemming 1.8 includes an inner layer 1.7 made of linen/flax material and an outer layer 1.9 made of polyester fabrics or polyester super soft fabric material and arranged outside the inner layer 1.7.

The three-dimensional breathable base layer 1 is fixedly connected to the three-dimensional weaved layer 2, and the three-dimensional breathable base layer 1 is directly sewn to the lower side of the three-dimensional weaved layer 2 through cotton threads.

Embodiment 2: On the basis of Embodiment 1, the three-dimensional breathable base layer 1 and the three-dimensional weaved layer 2 are detachably connected to each other. The three-dimensional breathable base layer 1 is provided with a Velcro fleece layer, and the three-dimensional weaved layer 2 is provided with a Velcro hook layer.

Embodiment 3: On the basis of Embodiment 1, the three-dimensional breathable base layer 1 and the three-dimensional weaved layer 2 are detachably connected to each other. The three-dimensional breathable base layer 1 and the three-dimensional weaved layer 2 are connected through press studs/button. Female buttons are provided on the upper side of the three-dimensional breathable base layer 1, and male buttons are provided on the lower side of the three-dimensional weaved layer 2.

Embodiment 4: On the basis of Embodiment 1, the three-dimensional breathable base layer 1 and the three-dimensional weaved layer 2 are detachably connected. The three-dimensional breathable base layer 1 is provided with button holes, and the three-dimensional weaved layer 2 is provided with buttons.

In this application, by arranging a three-dimensional breathable base layer 1 under the three-dimensional weaved layer 2 of chenille threads, it can not only increase the air permeability, but also make the distance between the three-dimensional weaved layer 2 and the ground farther, reducing the affection of the moisture and water vapor on the ground to the three-dimensional braid layer 2. The mesh size of the bottom layer 1.1 is smaller than the mesh size of the top layer 1.3, which can not only reduce the influence of ground moisture, but also play a certain role in isolating debris on the ground. There are evenly distributed glue dots 1.5 on the lower surface of the bottom layer 1.1, which can play an anti-slip role and prevent the carpet from sliding on the ground due to the user's actions after the carpet is laid on the ground. The three-dimensional support layer 1.2 is made of nylon coils with lower and upper ends connected to the bottom layer 1.1 and the top layer 1.3 respectively. When the three-dimensional support layer 1.2 is under pressure, the nylon coils can absorb the pressure and deform, making the carpet of the present disclosure softer and making the user's feet feel more comfortable. The three-dimensional weaved layer 2 is woven with chenille threads using plain knitting needles. The three-dimensional weaved layer 2 is softer and has better air permeability, and the diameter of the chenille threads is not less than 1 cm, so when the chenille threads are interlaced/weaved, gaps will be formed, which provides a better air permeability. Moreover, when the chenille threads are interlaced, the overall height of the three-dimensional weaved layer 2 of chenille threads will be larger, making the feet feel softer. Since the three-dimensional weaved layer 2 has gaps, the three-dimensional breathable base layer 1 and the three-dimensional weaved layer 2 can be sewn together, and debris can be easily poured out. Of course, in order to make the carpet in the present disclosure easier to be cleaned, the three-dimensional breathable base layer 1 and the three-dimensional weaved layer 2 may be detachably connected, thus the three-dimensional weaved layer 2 can be disassembled and cleaned.

What is claimed is:

1. A chenille carpet, comprising a three-dimensional breathable base layer (1), wherein a three-dimensional weaved layer (2) made of chenille threads is provided on the three-dimensional breathable base layer (1), and the chenille threads in the three-dimensional weaved layer (2) have a diameter not less than 1 cm.

2. The chenille carpet according to claim 1, wherein the three-dimensional weaved layer (2) is weaved by plain knitting needles using chenille threads.

3. The chenille carpet according to claim 1, wherein the three-dimensional breathable base layer (1) comprises a bottom layer (1.1), a three-dimensional support layer (1.2) provided on the bottom layer (1.1), and a top layer (1.3) provided on the three-dimensional support layer (1.2), a hemming (1.8) is provided on an edge of the base layer (1), and the bottom layer (1.1) and the top layer (1.3) are mesh cloth.

4. The chenille carpet according to claim 3, wherein a mesh size of the bottom layer (1.1) is less than a mesh size of the top layer (1.3), and the bottom layer (1.1) is provided with evenly distributed glue dots (1.5) at its bottom surface.

5. The chenille carpet according to claim 3, wherein the three-dimensional support layer (1.2) is made of nylon coils with lower and upper ends connected to the bottom layer (1.1) and the top layer (1.3) respectively.

6. The chenille carpet according to claim 3, wherein the hemming (1.8) comprises an inner layer (1.7) made of linen material and an outer layer (1.9) provided on the inner layer (1.7) and made of polyester fabrics or polyester super soft fabric material.

7. The chenille carpet according to claim 1, wherein the three-dimensional breathable base layer (1) and the three-dimensional weaved layer (2) are detachably connected.

8. The chenille carpet according to claim 1, wherein the three-dimensional breathable base layer (1) and the three-dimensional weaved layer (2) are fixedly connected.
